(19) United States
(12) Patent Application Publication
(10) Pub. No.: US 2026/0201472 A1
(43) Pub. Date: Jul. 16, 2026

(54) Methods of Screening and Compounds

(71) Applicant: PXD Limited, Newcastle upon Tyne (GB)

(72) Inventor: David Langton, Newcastle upon Tyne (GB)

(21) Appl. No.: 19/134,594

(22) PCT Filed: Nov. 30, 2023
(86) PCT No.: PCT/GB2023/053103

§ 371 Date: May 30, 2025

(30) Foreign Application Priority Data
Dec. 2, 2022 (GB) 2218169.7
Dec. 15, 2022 (GB) 2218967.4

(51) Int. Cl. C12Q 1/6883; G01N 33/68

(52) U.S. Cl. CPC C12Q 1/6883; G01N 33/6893; C12Q 2600/118; C12Q 2600/156; G01N 2500/10

(57) ABSTRACT

Methods of screening a subject to determine whether or not they are at risk of having an adverse reaction to metal debris (ARMD), also known as adverse local tissue reaction (ALTR), methods of screening compounds for use in preventing or ameliorating such an adverse response and compounds for use in treating a subject.

The present invention relates to methods of screening a subject to determine whether or not they are at risk of having an adverse reaction to metal debris (ARMD), also known as adverse local tissue reaction (ALTR), methods of screening compounds for use in preventing or ameliorating such an adverse response and compounds for use in treating a subject. An example of such a reaction includes an aseptic lymphocyte dominated vasculitis associated lesion (ALVAL). In particular, the present invention relates to determining whether or not a subject is at risk of having an adverse reaction to an implant.

Hip replacement or hip arthroplasty is a procedure in which the hip joint is replaced by a prosthetic implant. A total hip replacement consists of replacing the acetabulum and the femoral head while hemiarthroplasty only replaces the femoral head.

Metal on metal (MoM) hip replacements were reintroduced globally at the turn of the century (Treacy R B, McBryde C W, Pynsent P B. Birmingham hip resurfacing arthroplasty. A minimum follow-up of five years. J Bone Joint Surg Br. 2005; 87 (2): 167-70). With promise of increased stability (lower risk of dislocation) and a reduction in wear, they quickly gained popularity with surgeons throughout the world (12th Annual Report. National Joint Registry of England and Wales. 2015.). Intended to be more durable than conventional plastic hips, they were implanted mainly into younger subjects in order to allow them to return to as active a life as possible (McMinn D, Daniel J. History and modern concepts in surface replacement. Proc Inst Mech Eng H. 2006; 220 (2): 239-51).

Unfortunately, complications began to emerge. This was primarily due to adverse immune responses that subjects developed to metal debris (primarily composed of cobalt and chromium particles) which was generated from the hips (Pandit H, Glyn-Jones S, McLardy-Smith P, Gundle R, Whitwell D, Gibbons C L, et al. Pseudotumours associated with metal-on-metal hip resurfacings. J Bone Joint Surg Br. 2008; 90 (7): 847-51).

There appear to be two general cellular responses (Natu S, Sidaginamale R P, Gandhi J, Langton D J, Nargol A V. Adverse reactions to metal debris: histopathological features of periprosthetic soft tissue reactions seen in association with failed metal on metal hip arthroplasties. J Clin Pathol. 2012; 65 (5): 409-18.). With massive metal exposure, the predominant cellular response is macrophagic, and the resulting injury is mostly limited to the bone. (10) With intermediate levels of metal exposure, a cellular response (known as aseptic lymphocyte dominated vasculitis associated lesion (“ALVAL”) (11) can develop alongside the macrophagic infiltration. ALVAL is associated with the development of massive fluid effusions and widespread soft tissue necrosis (Langton D J, Jameson S S, Joyce T J, Hallab N J, Natu S, Nargol A V. Early failure of metal-on-metal bearings in hip resurfacing and large-diameter total hip replacement: A consequence of excess wear. J Bone Joint Surg Br. 2010; 92 (1): 38-46). The risk factors for the development of ALVAL are poorly understood. Unfortunately, ARMD/ALTR (Langton D J, Joyce T J, Jameson S S, Natu S, Holland J P, Nargol AVF, De Smet K. Adverse reaction to metal debris following hip resurfacing the influence of component type, orientation and volumetric wear. Journal of Bone and Joint Surgery (Br) 2011; 93:566; Langton D J, Jameson S S, Joyce T J, Natu S, Nargol AVF. Early failure of metal-on-metal bearings in hip resurfacing and large-diameter total hip replacement. A CONSEQUENCE O F EXCESS WEAR. Journal of Bone and Joint Surgery (Br) 2010; 92-B; and, Pseudotumours associated with metal-on-metal hip resurfacings. Pandit H, Glyn-Jones S, McLardy-Smith P, Gundle R, Whitwell D, Gibbons C L, Ostlere S, Athanasou N, Gill H S, Murray D W. J Bone Joint Surg Br. 2008 July; 90 (7): 847-51) is not limited to metal on metal devices (Jacobs J J, Cooper H J, Urban R M, Wixson R L, Della Valle C J. What do we know about taper corrosion in total hip arthroplasty? J Arthroplasty. 2014; 29 (4): 668-9). Over the last decade, there has been a global trend towards the adoption of the use of larger diameter bearings as standard, irrespective of the bearing combination (ie metal on plastic, ceramics) (12th Annual Report. National Joint Registry of England and Wales. 2015). During this time period, however, the support structure to accommodate these large diameter heads (the male taper/trunnion) was reduced in size (Langton D J, Sidaginamale R, Lord J K, Nargol A V, Joyce T J. Taper junction failure in large-diameter metal-on-metal bearings. Bone Joint Res. 2012; 1 (4): 56-63) The Applicant have observed several subjects who have suffered severe reactions from this interface-the “taper junction” (Langton D J, Jameson S S, Joyce T J, Gandhi J N, Sidaginamale R, Mereddy P, et al. Accelerating failure rate of the ASR total hip replacement. J Bone Joint Surg Br. 2011; 93 (8): 1011-6).

The Applicant believes that there will be a global issue with early failure of joint replacements over the next ten years. The Applicant has thus determined that the identification of subjects most at risk of developing ARMD/ALTR would be beneficial in order to streamline subject surveillance and/or consider alternative approaches in certain cases to avoid potentially catastrophic subject reactions. It may also facilitate the development of therapeutic interventions.

In accordance with the present invention, there is provided a method of screening a subject to determine the likelihood of adverse response to metal debris (ARMD) or adverse local tissue reaction (ALTR), said method comprising the steps of:
determining whether the subject has one or more of the following markers associated with an adverse response wherein the marker comprises one or more of the following SNPs: rs17090828,
GSA-rs1930426, GSA-rs79208627, GSA-rs45539538, GSA-rs76147841, GSA-rs117972780, rs4798850, rs11974031, rs9301947, GSA.rs17716559 PIGN, rs12248205, rs1392779, rs13145571, GSA-rs35269015, GSA-rs68164453, GSA-rs10985824, GSA-rs190079656, GSA-rs76147841, GSA-rs117972780, GSA-rs79208627, GSA-rs976508, GSA-rs35269015, GSA-rs76147841, GSA-rs1930426 and GSA-rs45539538, wherein the presence of one or more of the markers associated with an adverse response is indicative of an increased likelihood of an adverse response in a subject.

Adverse reaction to metal debris (ARMD) or adverse local tissue reaction (ALTR), such as ALVAL, is a newly described, pathological response that can occur in response to implanted metal devices, which is frequently associated with significant pain, disability and irreversible tissue destruction. It is a poorly understood condition, but one which may affect hundreds of thousands of subjects with metal implants throughout the world. The single nucleotide polymorphisms (SNPs) of the present invention have been identified by the Applicant as being associated with an adverse response to metal debris (ARMD) or adverse local tissue reaction (ALTR).

While the use of MoM hips has been greatly reduced in light of such complications, there is an increasing number of reports of ALVAL in MoP (metal on plastic) devices. It is possible that ARMD/ALTR may well be, and has been for decades, an under recognised source of unexplained pain and reduced subject satisfaction following joint replacement surgery. Improving the understanding of ARMD/ALTR is therefore important for the current management of at-risk subjects, for the future development of prostheses and, possibly, the investigation and treatment of other immune mediated/inflammatory conditions. Furthermore, it may open a new avenue for personalised joint replacement guided by an individual's genetically determined reactivity to certain compounds. As an example, despite the negative publicity, MoM hip resurfacing has performed extremely well in younger active male subjects.

There is also an increasing amount of evidence to indicate that ALVAL is under recognised in total knee replacements. Kilb et al and Lombardi have reported that between 7 and 44% of failed TKRs show histological evidence of ALVAL (Impact of perivascular lymphocytic infiltration in aseptic total knee revision. Crawford D A, Passias B J, Adams J B, Berend K R, Lombardi A V. Bone Joint J. 2021 June;103-B (6 Supple A): 145-149; Perivascular lymphocytic infiltration is not limited to metal-on-metal bearings. Ng V Y, Lombardi A V Jr, Berend K R, Skeels M D, Adams J B. Clin Orthop Relat Res. 2011 February;469 (2): 523-9; Kilb BKJ, Kurmis A P, Parry M, Sherwood K, Keown P, Masri B A, Duncan C P, Garbuz D S. Frank Stinchfield Award: Identification of the At-risk Genotype for Development of Pseudotumors Around Metal-on-metal THAs. Clin Orthop Relat Res. 2018 February;476 (2): 230-241. doi:)

The method of the present invention may be carried out before a medical procedure, so-called “pre-operation” or “pre-op” and/or after an operation, so-called “post-operation” or “post-op”.

The method of the present invention may be employed for use in any implant procedure, including hip replacement, cardiac stents, knees, shoulders, elbows and the like. The present invention may be employed in procedures requiring an implant.

Advantageously, identifying the presence of an increased risk of an adverse response to metal debris (ARMD) or adverse local tissue reaction (ALTR) in a subject allows health care professionals to more closely monitor and/or administer treatment sooner in high risk individuals, improving the likelihood of recovery in such individuals.

Adverse reactions can occur with materials comprising one or more of the following or ions thereof: Co, Cr, Zr, Ti, nickel, aluminium and/or polyethylene.

The marker associated with ARMD/ALTR may comprise any one or more of the following: HLA genotypes may comprise any one or more of the following such as HLA-DRB1*01, DRB1*04, DRB1*10 (18, 19) DQA1*05: 01/DQB1*02:01 HLA-DRB1*07, HLA-DRB1*0103, HLA-DRB1*04 and HLA-DRB3*0301. (20) and/or any one or more from the following table:

| :SNP | Gene | Protein |
|---|---|---|
| GSA-rs35269015 | MCC | relating to the function and/or |
| | | expression of MCC |
| GSA-rs68164453 | Intragenic | relating to the function and/or |
| | | expression of LOC105374976 |
| GSA-rs10985824 | u/s RABGAP1 | relating to the function and/or |
| | | expression of RABGAP1 |
| GSA-rs190079656 | ADAMTSL3 | relating to the function and/or |
| | | expression of the ADAMTSL3 |
| GSA-rs117972780 | OR1L4 | relating to the function and/or |
| | | expression of OR1L4 |
| GSA-rs45539538 | CYP24A1 | relating to the function and/or |
| | | expression of CYP24A1 |
| GSA-rs79208627 | LOC101927964 | relating to the function and/or |
| | | expression of LOC101927964 |
| GSA-rs976508 | PIGN | relating to the function and/or |
| | | expression of PIGN |
| GSA-rs76147841 | GABBR2 | relating to the function and/or |
| | | expression of GABBR2 |
| GSA-rs1930426 | u/s RABGAP1 | relating to the function and/or |
| | | expression of GABBR2 |
| rs56177975 | d/s TGFB2 | relating to the function and/or |
| | | expression of TGF2 |
| rs1392779 | between INPP4B | (relating to the function and/or |
| | and IL5 | expression of INPP4B and IL5 |
| rs13145571 | SULT1E1 | relating to the function and/or |
| | | expression of SULT1E1 |
| rs10790129 | intergenic | |
| rs2304968 | SLFN12L | relating to the function and/or |
| | | expression of SLFN12L |
| rs4798850 | RAB31 | relating to the function and/or |
| | | expression of RAB31 |
| rs57039293 | UMAD1 | Relating to the function and/or |
| | | expression of UMAD1 |
| rs79000696 | CYP24A1 | Relating to the function and/or |
| | | expression of CYP24A1 |

ALVAL is a lymphocyte driven response and is associated with CoCr alloys, rarely with non-cobalt containing components. ALVAL can result in widespread, irreversible tissue damage.

When metal particles are liberated from prostheses they combine with carrier proteins to form metalloproteins. Metalloproteins are ingested by dendritic cells or macrophages in a process called “phagocytosis”. After phagocytosis, the particle is transported to a lysosome. There, it is acidified, and the particle fragments into its constituent peptide chains. At the lysosome, a structure called the major histocompatibility complex (MHC) can bind with one of these peptides. It does this at a part of its structure called the “peptide binding groove”.

After an MHC molecule has bound a fragment with sufficient stability, the MHC molecule and the newly bound peptide travel to the surface of the cell and “present” the peptide to the outside environment (for this reason, macrophages and dendritic cells are called “antigen presenting cells”). Here, any passing lymphocytes may bind this complex and then become activated, releasing cytokines and travelling to the source of antigen release. This lymphocyte “activation” is the key step in determining whether ALVAL develops.

The three dimensional structure of the peptide binding groove is critical in determining which peptides are “presented”. This structure is genetically encoded. Some individuals have genes which encode very different peptide binding grooves-thus some individuals will respond differently to different antigens.

The marker associated with adverse reaction to metal debris (ARMD) and/or adverse local tissue reaction (ALTR) may comprise a HLA genotype, a subject's age, a subject's sex, vitamin D concentration and/or blood concentration of and/or sensitivity to cobalt, chromium, titanium, polyethylene, polyurethane, polytetrafluoroethylene, polyester, polyoxymethylene and/or zirconium.

Advantageously, the at least one surrogate marker is the latitude of the individual's residence and vitamin D status is estimated to be low where the latitude of the individual's residence is above 35° North or below 35° South.

Advantageously, the use of surrogate markers to estimate vitamin D status in an individual provides a low cost and readily accessible means of estimating vitamin D status. It is readily acknowledged that at these latitudes, individuals are less likely to be exposed to sufficient UVB radiation to produce the required levels of vitamin D. As dietary vitamin D intake is typically low, individuals' resident at these latitudes are likely to be deficient in vitamin D for at least part of the year.

Optionally, a second surrogate marker is calendar month at the time of carrying out the method and vitamin D status is estimated to be low where the latitude of the individual's residence is above 35° North and the calendar month is between October and April or where the latitude of the individual's residence is below 35° South and the calendar month is between May and September.

Advantageously, taking into account the latitude and season when estimating vitamin D status provides a more accurate estimate of vitamin D status as individuals resident above 35° North or below 35° South are more likely to be vitamin D deficient in the winter months when daylight hours and therefore UVB exposure are reduced.

Optionally, a second surrogate marker is the individual's ethnicity and vitamin D status is estimated to be low where the latitude of the individual's residence is above 35° North and the individual has African, African-Caribbean or South Asian ethnicity or where the latitude of the individual's residence is below 35° South and the individual has African, African-Caribbean or South Asian ethnicity.

Advantageously, taking into account the latitude and ethnicity when estimating vitamin D status provides a more accurate estimate of vitamin D status as individuals with African, African-Caribbean or South Asian ethnicity who are resident above 35° North or below 35° South are more likely to be vitamin D deficient as sunlight, and therefore UVB absorption, through the skin is reduced.

Optionally, the at least one surrogate marker is the individual's BMI and vitamin D status is estimated to be low where the individual has a BMI over 25.

Advantageously, considering BMI when estimating an individual's vitamin D status may provide a more accurate vitamin D status estimate as those with an elevated BMI are more likely to have a low vitamin D status.

Optionally, the at least one surrogate marker is the individual's socioeconomic status and vitamin D status is estimated to be low where the individual has a low socioeconomic status.

Advantageously, considering socioeconomic status when estimating an individual's vitamin D status may provide a more accurate vitamin D status estimate as those with a low socioeconomic status are more likely to have a low vitamin D status.

Optionally, socioeconomic status is determined by considering one or more of the following factors: household income, occupation, level of education, place of residence. Socioeconomic status is estimated to be low when one or more of the above factors are considered to be of a lower level or value than the average in the population.

Preferably, vitamin D levels are determined by measuring the level of circulating 25-hydroxyvitamin D and the predetermined threshold is most preferably 75 nmol/L; in some embodiments 30 nmol/L; and in alternative embodiments 20 nmol/L.

Advantageously, levels of circulating 25-hydroxyvitamin D below 75 nmol/L are considered to be low.

Optionally, the method further includes the step of determining the gender of the individual.

The biological sample may comprise a solid and/or fluid sample. The fluid sample may be a blood sample, saliva or a blood extract sample. The biological sample may comprise skin cells from the buccal cavity, for example.

The method may be an in vitro method.

The method may be employed to be used prior to joint replacement to determine risk of subject developing ARMD/ALTR related failure and/or used for joints in situ, in combination with blood metal ion concentrations to determine future risk of ARMD/ALTR related failure.

The method may comprise the step of treating a subject to prevent an adverse reaction to metal debris (ARMD) and/or adverse local tissue reaction (ALTR) to an implant comprising administering an agent to ameliorate or prevent an adverse reaction.

In accordance with another aspect of the present invention, there is provided a method of screening for a compound for use in preventing or ameliorating the likelihood of adverse response to metal debris (ARMD) or adverse local tissue reaction (ALTR) in a subject, said method comprising the steps of identifying an compound that interferes with MHC mediated immune response.

In accordance with a further aspect of the present invention, there is provided a composition for use preventing or ameliorating the likelihood of adverse response to metal debris (ARMD) or adverse local tissue reaction (ALTR) in a subject.

The composition may comprise testosterone or a metabolic precursor thereof.

Preferably the sample is a whole blood sample.

The Applicant has reverse engineered thousands of explanted prostheses to determine the amount of wear that has occurred in the body. The total amount of material lost is termed “volumetric wear”.

The Applicant used existing software which computer models the resulting peptide binding groove shape if the DQA1/DQB1 genetic combination is inputted. The software predicted the strength of binding of various peptide fragments to each possible peptide binding groove combination. The Applicant's discovered that subjects with genotypes suited to the N terminal peptide fragments of albumin were significantly more likely to develop ALVAL. Other genotypes were associated with pain in the presence of a macrophage only ARMD response.

Using regression statistical modelling, with subject age and sex as variables included, the Applicant has invented a means to estimate a subject's relative risk of developing ARMD (either macrophage dominated or lymphocyte dominated (ALVAL). This could be used post operatively for advising follow up strategy but also pre-operatively to guide implant selection.

HLA genes (specifically the class II genes DQA1/DQB1) and the SNPs can be collected using non-invasive methods, such as from a cheek swab.

It is highly likely that the subjects mounting an ALVAL response develop antibodies to the “antigen”. Knowing that the antigen will have particular affinity to the genetically determined peptide binding grooves will permit the use of a blood test to determine whether ALVAL is active in at risk subjects. The methods of the present invention may comprise the use of enzyme-linked immunosorbent assay (ELISA).

According to a second aspect of the present invention, there is provided a kit for screening a subject to determine the likelihood of adverse response to metal debris (ARMD) or adverse local tissue reaction (ALTR), comprising: reagents for determining whether an individual has any of the following SNPs: rs17090828, GSA-rs1930426, GSA-rs79208627, GSA-rs45539538, GSA-rs76147841, GSA-rs117972780, rs4798850, rs11974031, rs9301947, GSA.rs17716559 PIGN, rs12248205, rs1392779, rs13145571, GSA-rs35269015, GSA-rs68164453, GSA-rs10985824, GSA-rs190079656, GSA-rs76147841, GSA-rs117972780, GSA-rs79208627, GSA-rs976508, GSA-rs35269015, GSA-rs76147841, GSA-rs1930426,GSA-rs45539538 and rs79000696; rs57039293 and optionally reagents for determining vitamin D status of a subject; and directions for determining the likelihood of adverse response to metal debris (ARMD) or adverse local tissue reaction (ALTR) based on the presence of any of the SNPs and optionally, the vitamin D status of the subject.

Optionally, the reagents for determining whether a subject has any of the marker SNPs comprises at least one set of oligonucleotide primers.

Advantageously, oligonucleotide primers provide a means of identifying the presence of any of the marker SNPs by standard molecular biological techniques such as PCR.

According to a third aspect of the present invention, there is provided a device for screening a subject to determine the likelihood of adverse response to metal debris (ARMD) or adverse local tissue reaction (ALTR) comprising: a means of inputting a biological sample from the subject; a means of determining whether the sample comprises any of the following SNPs: rs17090828, GSA-rs1930426, GSA-rs79208627, GSA-rs45539538, GSA-rs76147841, GSA-rs117972780, rs4798850, rs11974031, rs9301947, GSA.rs17716559 PIGN, rs12248205, rs1392779, rs13145571, GSA-rs35269015, GSA-rs68164453, GSA-rs10985824, GSA-rs190079656, GSA-rs76147841, GSA-rs117972780, GSA-rs79208627, GSA-rs976508, GSA-rs35269015, GSA-rs76147841, GSA-rs1930426 and GSA-rs45539538; and rs79000696; rs57039293 optionally, a means of determining the vitamin D levels of the sample or a means of inputting the subject's vitamin D levels or an estimate of the individual's vitamin D status.

Optionally, the biological sample is subject to pre-processing steps before being inputted into the device.

Optionally, the biological sample is a biological sample selected from the following: whole blood sample, blood serum sample, blood plasma sample, urine sample, saliva sample, buccal swab.

Preferably, the device further comprises a means for displaying output data.

Advantageously, a means for displaying output data allows the operator of the device to readily access the data.

Optionally, the means for displaying output data display is a display integrated into the device.

Optionally, the means for displaying output data is an external display such as a computer, mobile phone or tablet device.

Advantageously, this allows the data to be accessed directly from a device such as a clinician's computer.

Optionally, the output data comprises an indication of whether an individual has at least one of the marker SNPs.

Optionally, the output data indicates a subject's vitamin D status.

Advantageously, this allows an operator to access the data relating to the individual's vitamin D status quickly and readily. This data can be used to determine the likelihood of adverse response to metal debris (ARMD) or adverse local tissue reaction (ALTR) of a subject.

Optionally, the output data indicates the likelihood of adverse response to metal debris (ARMD) or adverse local tissue reaction (ALTR) of a subject.

Optionally, the means of determining whether a subject has at least one of the marker SNPs comprises a molecular HLA assay.

Optionally, the means of determining whether a subject has at least one of the marker SNPs comprises a PCR assay.

Optionally, the means of determining vitamin D level comprises a lateral flow test.

Advantageously, a lateral flow test is a fast and cost effective means of determining vitamin D level in a subject.

Preferably, device is a point of care device.

Advantageously, a point of care device can be used to deliver a result at the point of care and therefore the subject can obtain a fast indication of their risk.

Preferably, the device is a microfluidic device. Advantageously, microfluidic devices only require a very small sample size meaning that a less invasive sample obtaining method can be used, i.e. a finger prick blood sample.

Vitamin D levels may be determined by measuring the level of circulating 25-hydroxyvitamin D and the predetermined threshold is most preferably 75 nmol/L; in some alternatives 30 nmol/L; and in other embodiments 20 nmol/L.

In accordance with a further aspect of the present invention, there is provided a method of assessing the risk of an individual developing adverse response to metal debris (ARMD) or adverse local tissue reaction (ALTR), the method comprising determining whether the individual has a genetic marker for metal sensitivity.

The genetic marker may comprise any one or more of the SNP markers described hereinabove and/or HLA haplotypes.

Unless defined otherwise, all technical and scientific terms used herein have the meaning commonly understood by a person skilled in the art to which this invention belongs.

To assist the reader, the following terms have the meanings ascribed to them below, unless specified otherwise.

The term “determining” refers to the collection of data and/or information from any source. For example, the data and/or information can be collected by primary means, i.e. by carrying out an assay on a biological sample from an individual to determine the presence or absence of the marker SNPs. The data and/or information can also be collected from secondary sources, i.e. by retrieving the HLA haplotype from the individual's medical records.

The term “body mass index” or “BMI” refers to a value derived from the mass and height of an individual. BMI can be calculated by dividing the weight of an individual in kilograms divided by their height in metres squared (BMI=mass (kg)/(height (meters) 2)). The skilled person would understand that any suitable alternative measure could be used to indicate an individual's weight normalised according to their height.

The nomenclature of the HLA system used throughout the present specification is that formally established by the World Health Organization HLA Nomenclature Committee.

The present invention will now be described, by way of example only, with reference to the accompanying examples and figures, in which:

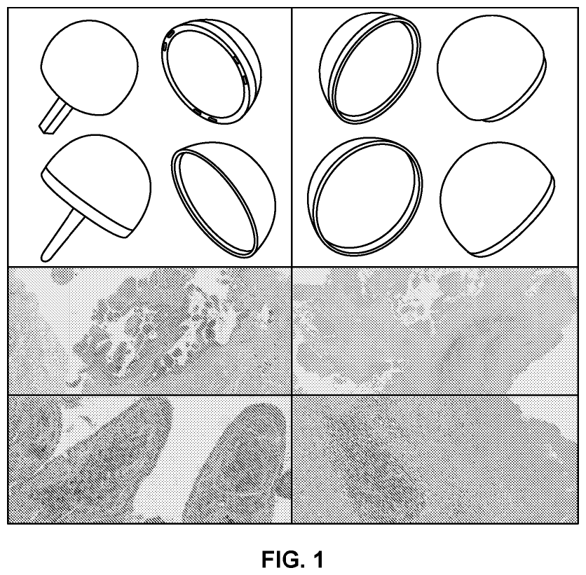

FIG. 1 shows an explant analysis and tissue responses of MoM hip components manufactured from CoCr alloy;

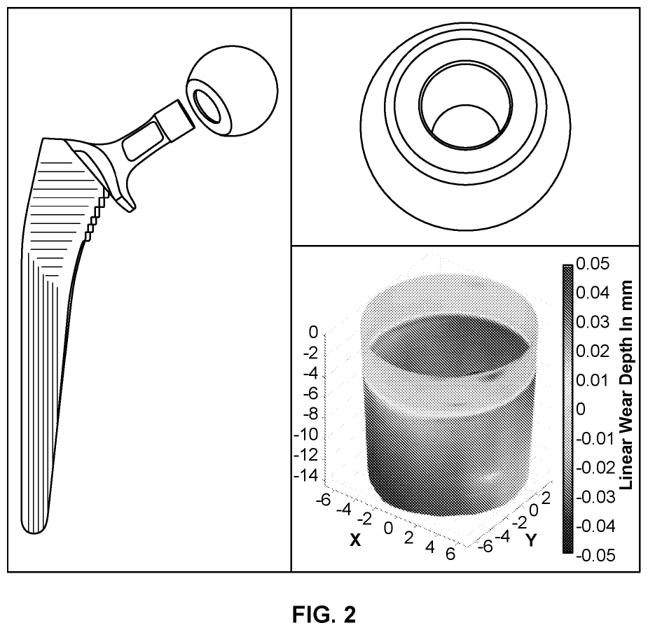

FIG. 2 shows the structure of total hip replacements (THRs);

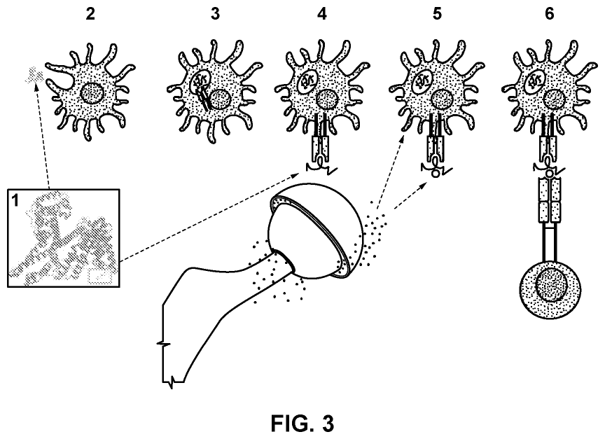

FIG. 3 Shows the pathogenesis of DTH;

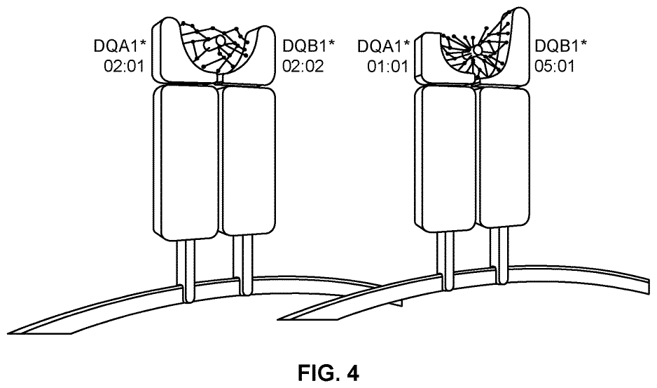

FIG. 4 is an illustration showing MHC structures and peptide presentation;

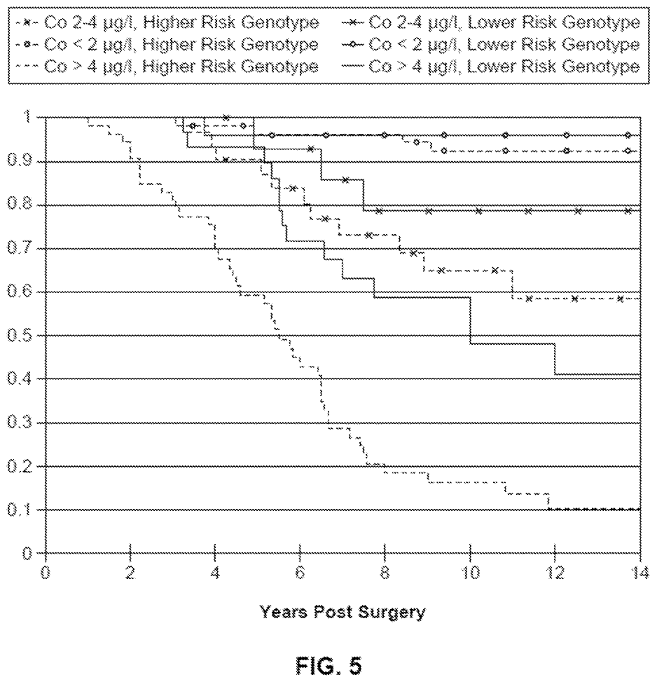

FIG. 5 is a Kaplan Meier survival analysis for all female subjects in the study. Failure secondary to ALVAL (mild/moderate/severe). The subjects have been sub-grouped according to their HLA genotypes and blood Co concentration. Higher risk genotype defined by the top 50% NTS binders, lower risk by the lowest 50%. Cox proportional hazards modelling using “Co<2, lower risk genotype” as the reference group returned hazard ratios (HR) (95% CI) of 1.95 (0.22-17.0) for Co<2, higher risk genotype; 5.95 (0.62-57.2) for Co 2-4, lower risk genotype; 12.54 (1.62-97.2) for Co 2-4, higher risk genotype; 18.7 (2.45-142) for Co>4, lower risk genotype; 53.1 (7.26-388) for Co>4, higher risk genotype;

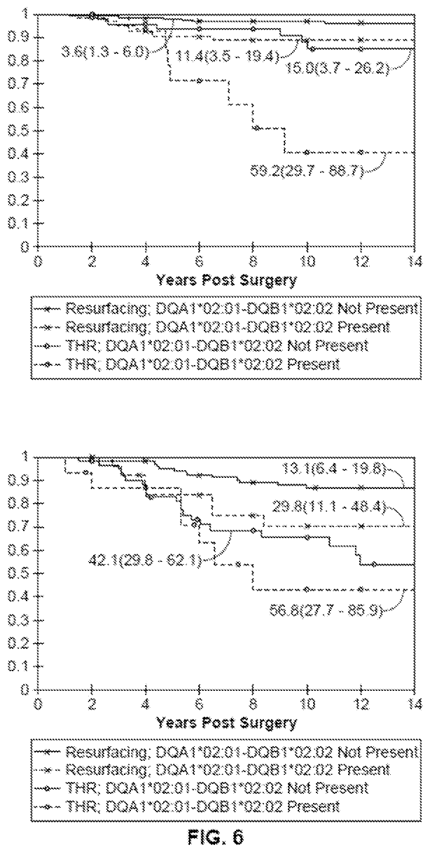

FIG. 6 is a Kaplan Meier survival analysis including all subjects in the study; Male subjects on the left, female subjects on the right. Failure defined as revision secondary to ALVAL (moderate or severe). The subjects have been sub divided according to implant type (hip resurfacing or THR) and genotype (possession of HLA-DQA1*02:01-DQB1*02:02). Failure rates at 12 years are shown at the bottom right of the charts (with 95% confidence intervals);

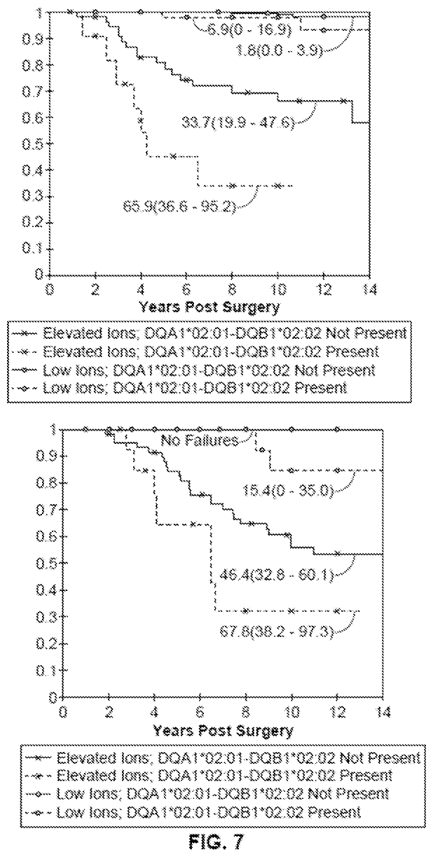

FIG. 7 is a Kaplan Meier survival analysis of the hip resurfacing subjects. Male subjects on left, female subjects on right. Failure defined as revision secondary to ALVAL (mild, moderate or severe). Subjects were subdivided according to blood ion concentrations: (“low” (Co<2; Cr<4) and “elevated” (Co≤2 or Cr≤4 μg/l)) and genotype (presence or absence of DQA1*02:01-DQB1*02:02). Failure rates at 12 years are shown on the bottom right of the charts (with 95% confidence intervals).

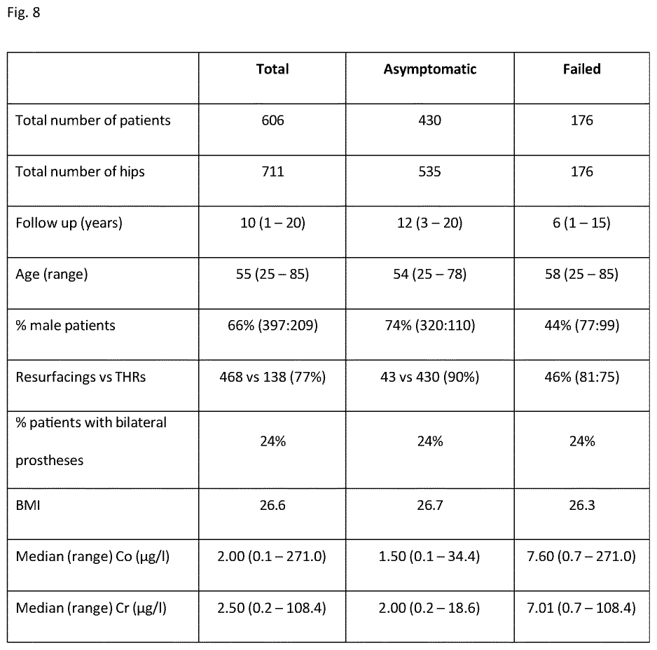

FIG. 8 is a table (Table 1) showing the clinical details of all subjects from all centres divided by clinical outcome;

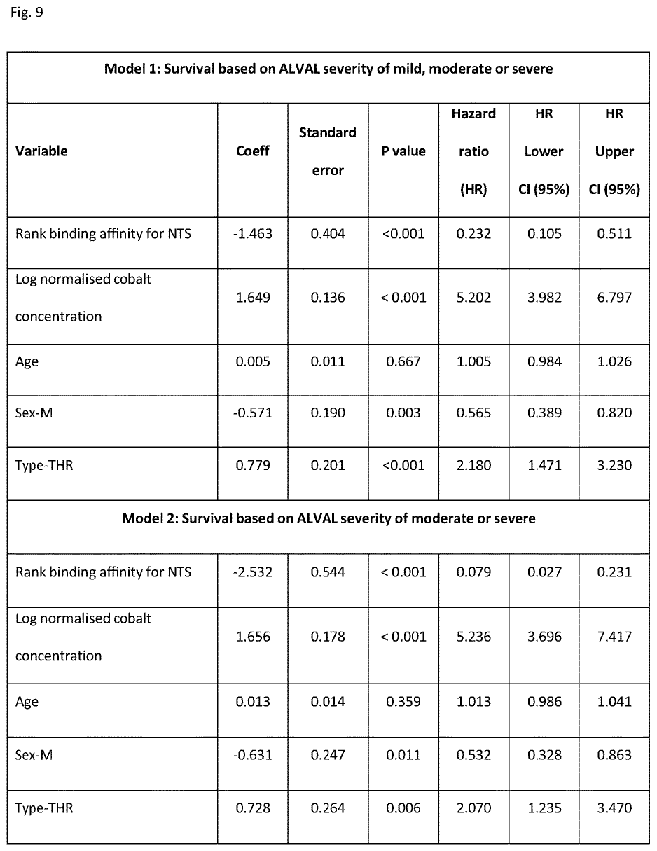

FIG. 9 is a table (Table 2) showing cox proportional hazards modelling of subjects studied; Cox proportional hazards modelling, all 606 patients involved in the study included. Patients were censored based on a minimum ALVAL grade of “mild” and above, then a second model was constructed with patients censored only if they had “moderate” ALVAL and above.

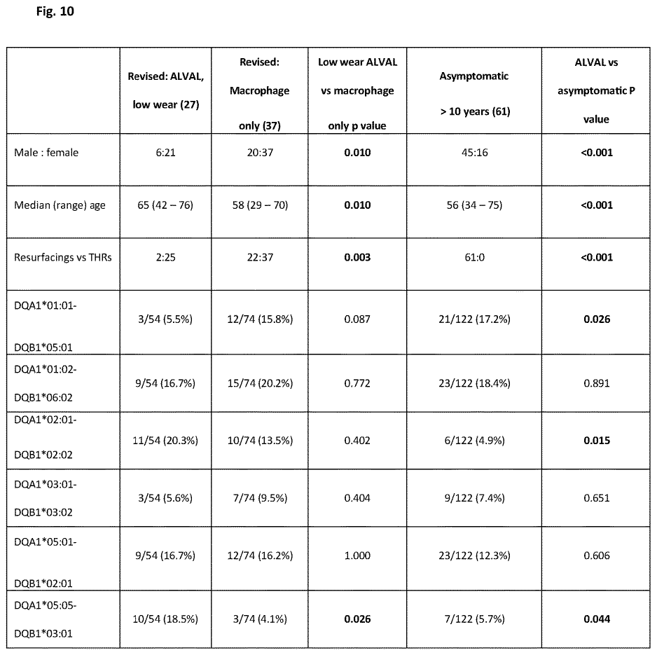

FIG. 10 is a table (Supplementary Data Table 1) showing extreme phenotype analysis where all class I and II HLA alleles were typed to six digit resolution. However, only four digit values were used in the comparisons between groups and in the modelling. The full set of allele frequency comparisons is shown in supplementary data table 1.;

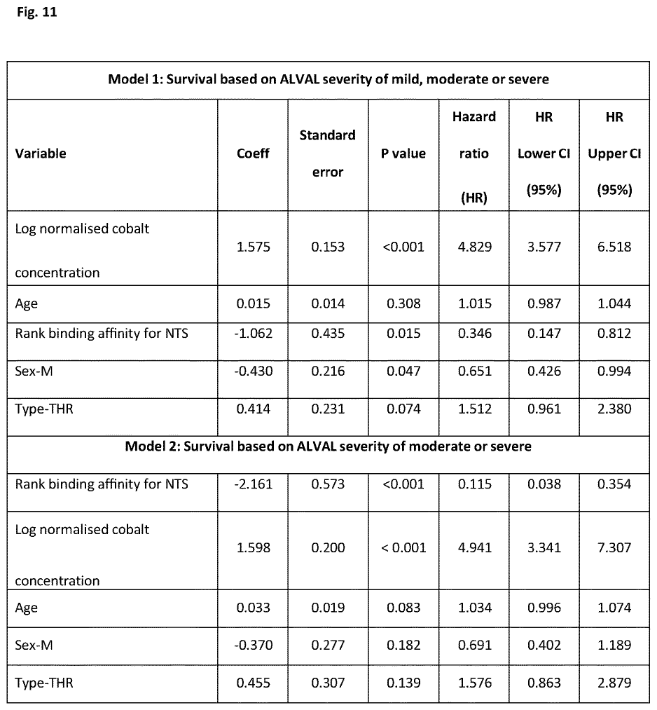

FIG. 11 is a table (Supplementary Data Table 2) showing Cox proportional hazards modelling from phase 1: centre 1 subjects. Cox proportional hazards modelling from phase 1: Centre 1 patients only, N=161.;

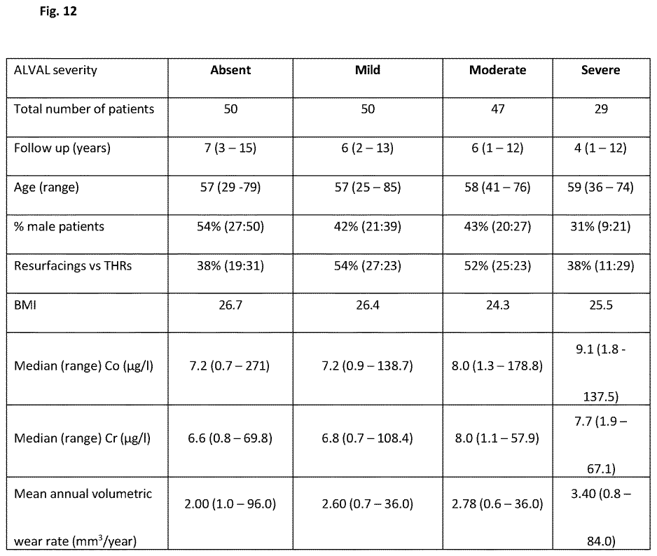

FIG. 12 is a table (Supplementary Data Table 3) showing clinical details of subjects who underwent revision of their prostheses;

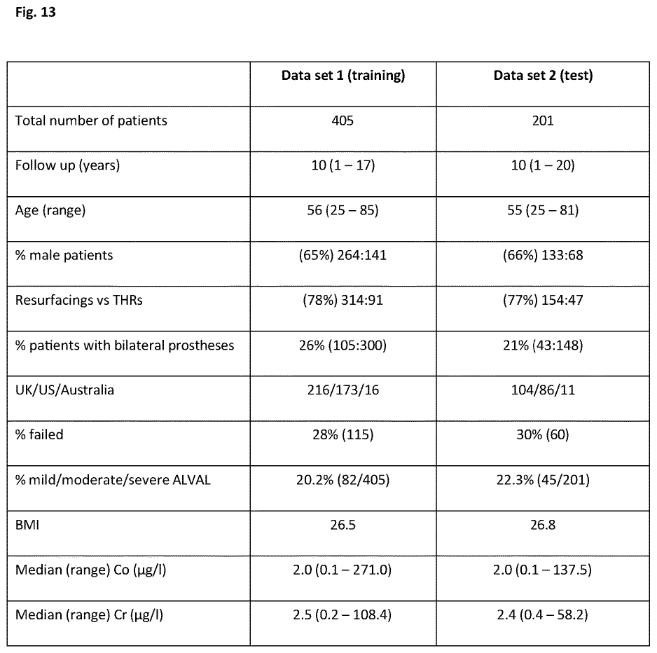

FIG. 13 is a table (Supplementary Data Table 4) showing demographics and clinical details of the training and validation datasets of the present invention;

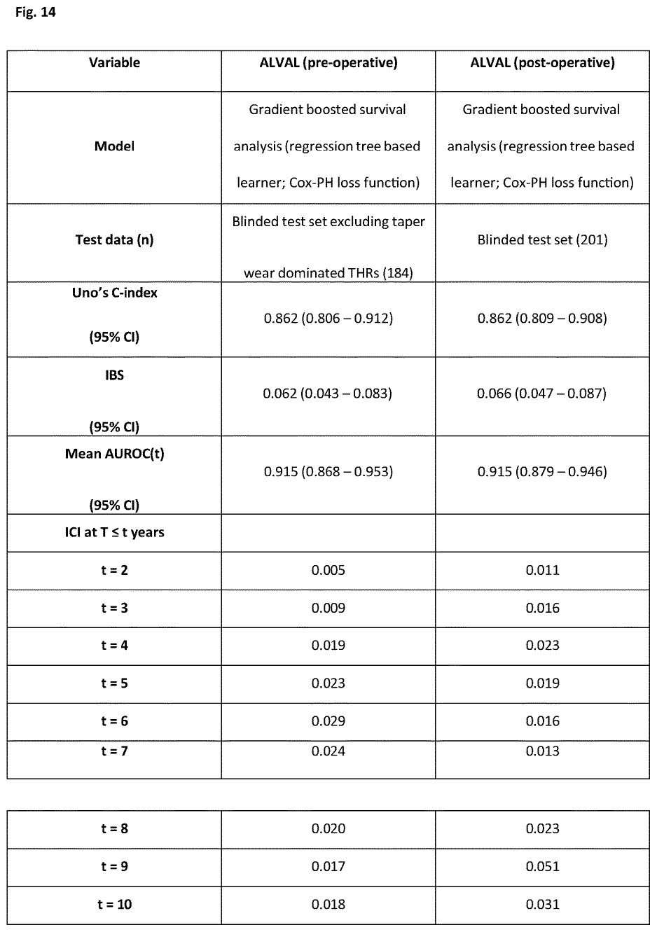

FIG. 14 is a table (Supplementary Data Table 5) showing the results of performance evaluation of the models on the test set. This table shows the results of performance evaluation of the presented models on the test set. Taper-dominated wearing THRs were excluded from the test set for the ALVAL pre-operative model to better fit the clinical context in which this model would be exposed to. As the resulting test sets were different for pre-operative and post-operative models, their results are not directly comparable. Similarly high performance was observed across each of the models.;

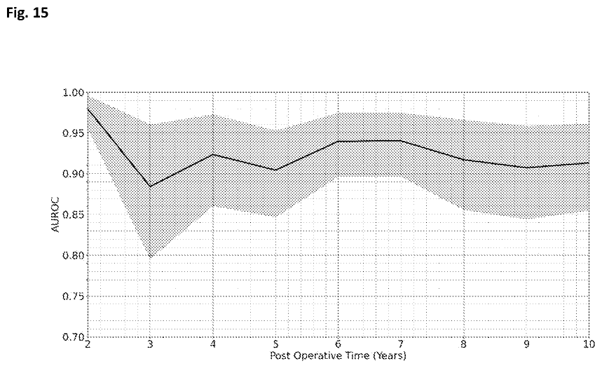

FIG. 15 is a time dependent AUROC (t) for the pre-operative model from 2 to 10 years after implantation (Supplementary Data FIG. 1). The shaded area represents the 95% confidence intervals calculated via the Bootstrap method.; and

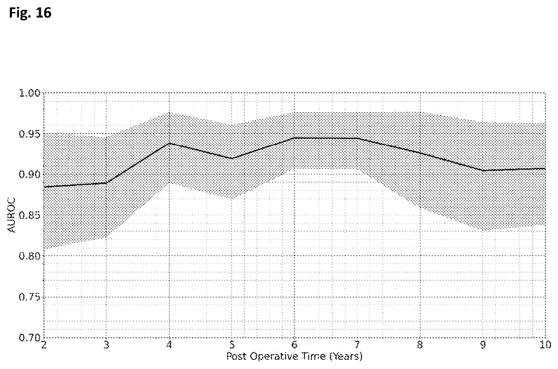

FIG. 16 is a time dependent AUROC (t) for the pre-operative model over time (Supplementary Data FIG. 2). The shaded area represents the 95% confidence intervals calculated via the Bootstrap method.

MoM hip components are manufactured from standard, medical grade CoCr alloy (ASTM-75 or ASTM-1537), which is composed of approximately 65% Co and 30% Cr by weight. Explanted prostheses can be reverse engineered using coordinate measuring machines (CMMs) to quantify the volumetric material loss through wear and corrosion (shown in red in the wear maps below). Serum or whole blood Co and Cr concentrations provide a reliable in-vivo surrogate measure of the rate of this material loss. The images below show (in descending order) the explanted prostheses, the corresponding wear maps, tissue sections at ×2 and ×15 magnification.

Left: Explanted Birmingham Hip Resurfacing with a volumetric wear rate of 25 mm3/year; blood Co=20.1 μg/l. There is a heavy macrophage infiltrate, but no lymphocytes are present and the synovial membrane remains largely intact (no ALVAL response observed). Right: Explanted ASR XL THR, with a volumetric wear rate of 1.5 mm3/year and a blood Co concentration=1.5 μg/l. There is a heavy perivascular lymphocyte infiltrate andextensive synovial necrosis (severe ALVAL).

In total hip arthroplasty (FIG. 2), the femoral neck is sectioned and a femoral stem (titanium, uncemented for the patients in this study) is placed down the femoral canal (left). The femoral head (top right) is press fit on to the stem, creating the “taper junction”. Metal debris can be generated from the taper junction, the great majority of which is released from the CoCr head. For the explanted THRs in this study, material loss from the bearing and tapers was quantified using a coordinate measuring machine. A taper wear map is shown (bottom right), with red areas indicating areas of material loss greater than 50 microns in depth.

FIG. 3 shows the pathogenesis of DTH.
1. The N terminal sequence (NTS) of albumin (red rectangle) contains two recognised metal binding sites.
2. Endogenous (self-proteins, including albumin) and exogenous (pathogens/ingested substances) proteins are ingested by APCs, which include macrophages and dendritic cells (purple).
3. The ingested proteins are acidified in the lysosomes of the APCs (29), where peptidases act to section the original proteins into their shorter, constituent peptides. These peptides compete for the binding groove of class II MHC complexes (red).
4. If a peptide forms a stable complex with the MHC molecule, the resulting MHC: peptide complex is transported to the cell surface
5. Self-peptides held in the binding groove of an MHC molecule can form complexes with metal ions (dark red sphere) released from implant surfaces (broken dark red arrow) to form haptens.
6. The MHC: peptide: metal complex may activate T4 lymphocytes resulting in sensitisation. This will happen if the lymphocyte has been activated via pattern recognition receptors (PRRs), which can occur due to local cellular damage through metal toxicity (broken red arrow).

FIG. 4 is a illustration showing the HLA-DQ molecule is an aß heterodimer of MHC class type II. The three dimensional shape of the peptide binding groove is formed by the combination of a and B chains, which are genetically encoded by the HLA-DQA1 and HLA-DQB1 alleles respectively. The structure of the peptide binding groove determines which peptides (foreign or self) are presented at the cell's surface [86], as is shown in the schematic below. As an example, in coeliac disease, patients possess HLA alleles which encode for peptide binding grooves with structures suited to the presentation of gluten

### EXAMPLES

### Methods: Subjects and Hospital Centres

Following Health Research Authority ethical approval (IRAS reference 227785), the study commenced at a single centre (centre 1, United Kingdom) where a large number of MoM hip arthroplasties were performed between 2002 and 2010. These subject cohorts have been described in full in previous publications. The subjects have been kept under surveillance with annual clinical review and blood metal ion testing. As part of an ethically approved project (IRAS reference 14119), subjects who undergo revision of their MoM hip prostheses have: undergone metal ion testing to assess Co and Cr concentrations in their blood, serum and hip joint synovial fluid samples; their explanted prostheses analysed to determine their volumetric wear; tissue samples excised at revision surgery assessed by a specialist histopathologist (SN). The total number of revision cases in the database at commencement of the current study was 420. Blood/serum metal ion testing. Applicant has carried out a substantial amount of work detailing the relationships between volumetric wear of implants and the corresponding concentrations of Co and Cr ion in the blood, serum and synovial fluid fractions. Samples were tested using this validated methodology. [20, 30]

### Wear Analysis.

Explanted prostheses were analysed using a coordinate measuring machine (Legex 322; Mitutoyo Ltd, Halifax, United Kingdom) to calculate the total amount of material that had been removed from the components in vivo. This material loss can be expressed in volumetric terms as ‘total volumetric wear’ (in mm3) or the total value can be divided by the number of years in vivo to provide a mean ‘volumetric wear rate’ (expressed in mm3/year). The accuracy of such methods has been validated and is of the order of 0.5 mm³ per component for bearings and 0.2 mm3 for tapers. [31] Throughout this paper, wear rates refer only to volumetric CoCr material loss.

For resurfacings, ‘total volumetric wear rates’ refer to the bearing surface wear rates (combined head and acetabular component volumetric wear rates). For THAs, ‘total volumetric wear rates’ refer to the combined wear rates of the bearings and female taper surface. THAs in the study were used with titanium stems. Yitanium release is small in comparison with CoCr. [32]

### Histopathological Tissue Assessment.

This was carried out as described in greater detail. Samples were taken from between two and four sites surrounding the implant. Up to ten paraffin blocks were processed per site to ensure the viable tissue was well represented. Samples were also sent to the microbiology department to identify sepsis. Adverse reaction to metal debris (ARMD) is an umbrella term which refers to clinical signs and symptoms occurring in association with metal debris. The typical response to metal debris is limited to a macrophage infiltrate. ALVAL is a subset of ARMD, referring to the additional lymphocyte infiltrate and histological features of DTH. The hallmark of ALVAL is the development of a perivascular lymphocytic infiltrate which appears to be dynamic process. Thin perivascular cuffs initially appear which increase in thickness as the recruitment of lymphocytes is further stimulated. These cuffs then either expand to develop into aggregates or coalesce into one another, forming larger aggregates. Once this process of lymphoid neogenesis is complete, pale germinal centres appear. The ALVAL response was graded from 0 (absent) to 3 (severe) according to the integrity of the synovial membrane and the stage of lymphocytic infiltration. This classification system has shown good intra and interobserver reliability.

### Part 1: Investigation of Genetic Associations Using Extreme Phenotype Group Comparison

The Applicant identified four groups of subjects, to represent the different phenotypes:
1. Subjects with joint failure who developed moderate/severe ALVAL in association with prostheses wearing at lower than the median wear rate of the total revision cohort.
2. Subjects with joint failure who developed moderate/severe ALVAL in association with prostheses wearing at greater than the median wear rate of the total revision cohort.
3. Subjects with joint failure with a pathological response limited to macrophage infiltration; no lymphocyte infiltration identified.
4. Subjects with joints remaining in situ. Subjects who were pain free and satisfied with the results of their hip arthroplasties at a minimum of ten years post surgery.

### DNA Sample Collection and Processing

A combination of ORAcollect OCR-100 buccal swabs and Oragene DNA OG-610 saliva collection kits (both DNA Genotek Inc, Ontario, Canada) were used to collect samples for DNA extraction. DNA was extracted from the swab and saliva samples using the Roche MagnaPure Compact automated platform (Roche Holding AG, Switzerland). DNA was then quantified using the Thermo Fisher Qubit dsDNA BR Assay kit (Thermo Fisher, Massachusetts, United States) and standardised to 25 ng/ul. HLA genotyping was performed using the One Lambda AllType NGS kits (One Lambda, USA), with the Illumina MiSeq platform (Illumina, USA). Full gene sequencing was carried out for HLA-A,-B,-C,-DQA1 and-DPA1 and partial gene sequencing (omission of exon 1) for HLA-DRB1,-DRB345,-DQB1 and-DPB1. HLA genotypes were analysed using One Lambda TypeStream Visual 1.3 software (One Lambda, USA).

Global locus-wise association for each HLA gene was performed using UNPHASED v 3.0.13. Haplotypes were estimated for DRB1-DQA1-DQB1 also in UNPHASED [36], and then the distribution of the HLA class I and II alleles were compared between groups using a standard approach.

### Part 2: In Silico Analysis of Peptide-HLA Class II Binding Affinity and Cox Proportional Hazards Modelling

ALVAL is indicated as being associated with the accumulation of large, albumin rich synovial fluid collections. Fluid taken from subjects with ALVAL also tends to contain a disproportionate elevation of metal ions in the synovial fluid compared to that which would be expected from the rate of material loss from the implant [33, 38]. This elevation is more pronounced with respect to Co ions in comparison to Cr. Furthermore, it is recognised that protein binding of metal ions delays their egression from the joint space, and that Co binds almost exclusively to albumin in synovial fluid and serum samples. [39, 40] Collectively, these observations imply that albumin bound Co is a key factor in the development of ALVAL.

### Candidate Antigen

The most likely candidate sequence we believed, would be the N-terminal sequence (NTS) which contains two metal ion binding sites. The first arises from the first triplet amino acid motif of albumin: Asp1-Ala2-His3 (the N terminal site). [41] The second (also termed site B), is partially composed of His9 and Asp13. The N terminal sequence is the most likely to be sectioned early in the cellular processing of albumin following its uptake by APCs. While site B exhibits greater binding affinity for Co, the N terminal site is formed from a singular domain, continuous amino acid sequence, not reliant on connections between domains to maintain its metal binding properties. [41]

### Peptide Binding Analysis

Validated software enables virtual construction of peptide binding grooves encoded by an individual's HLA genotype. This allows the calculation of the binding affinity between a particular HLA encoded peptide binding groove and an array of naturally occurring peptides. Using this software, in this investigation, we initially sought to: identify HLA genes associated with the development of ALVAL; determine whether HLA genes associated with the development of ALVAL at low rates of wear encode for peptide binding grooves with higher affinities for the N terminal metal binding sites of albumin.

Applicant performed in silico analysis of peptide-HLA class II binding affinity across HLA proteins found in the subject population. All HLA-DQA1,-DQB1, and DRB1 alleles were selected to assess the peptide binding affinity of their corresponding peptide binding proteins. HLA-DR is represented by HLA-DRA/DRB1 dimer. Since HLA-DRA is considered monomorphic, Applicant only used HLA-DRB1. HLA-DQ is represented by the HLA-DQA1/DQB1 dimer.

FASTA-formatted protein sequence data were retrieved from the UniProt database (www.uniprot.org) for human serum albumin (P02768). Applicant extracted the first 15 amino acids of the N terminal (DAHKSEVAHRFKDLG), a sequence which includes two recognised Co binding sites. Predictions for HLA binding to this sequence were performed using NetMHCIlpan4.0. [42] The rank binding affinities were calculated for all the possible DQ and DRB1 combinations. Applicant used the % EL rank score as the primary binding metric, as advised in personal communication with the software developers. [43] We investigated whether the binding scores influenced the risk of developing ALVAL over time using Cox proportional hazards modelling. Multiple survival models were constructed to explain the development of time dependent prosthetic failure associated with mild/moderate or severe ALVAL, using the following independent variables: NTS binding affinity; pre revision blood Co concentrations; pre revision blood Cr concentrations; subject sex; subject age at time of primary surgery; presence of bilateral prostheses; type of prosthesis (THR versus resurfacing arthroplasty).

Part 3: Expansion of Data Set, Inclusion of Subjects from Other Centres and Development of Machine Learning Algorithm

Applicant invited all remaining subjects in the database who had undergone revision surgery for whom there was a full complement of clinical data, including explanted components available for analysis. We also invited all remaining subjects under regular follow up who were recorded to be asymptomatic at greater than ten years follow up. Concurrently, Applicant expanded the study to include two other units. Centre 2 is a major specialist orthopaedic unit in New York, United States. Centre 3 is a teaching hospital and tertiary referral centre in Western Australia. The units manage the follow up of MoM subjects in a similar way and also routinely carry out analysis of explanted components. A similar research protocol was followed, with subjects who were asymptomatic as well as those who had experienced failure of their joints invited to give a sample for DNA analysis. The same parameters were recorded as at centre 1. When all samples had been analysed, the data set was randomly split 70/30, with the larger set used to train a machine learning algorithm for the prediction of the development of ALVAL. The remaining data was held back, blinded from the analysts and used to test the algorithm when it was finalised.

Two models were trained to predict hazard ratios and survival functions up to ten years after implantation of a MoM prosthesis for pre-operative and post-operative subjects:

1. Preoperative prediction of development of ALVAL. For this model, metal exposure was divided into two groups: low wear (Co concentrations stabilise to <2 μg/l) and increased wear (Co concentrations stabilise to ≥2 and ≤4 μg/l). 4 μg/l equates to approximately three times the wear rate of a well-functioning device. It was therefore not felt necessary to provide a preoperative prediction for metal concentrations above this level 2. Postoperative prediction of development of ALVAL, in which actual measured Co and Cr concentrations could be used in the modelling Statistics and machine learning approach to select a machine learning model which generalises well, one must disjoint the training and testing strategy. As the training and test set are assumed to be drawn from the same probability distribution, they should be identically distributed. Therefore, Applicant formulated our test set by randomly sampling the full dataset (without replacement) stratified on the event indicator. The training data comprised of the remaining samples. Feature engineering was carried out on the training data to identify features that best predicted risk of failure due to ARMD and ALVAL within ten years of implantation of a MoM prosthesis. Boruta [45], a random forest feature selection algorithm was applied to 2939 features, generated from a combination of: subject features; binding affinities of cis and trans haplotypes; binary presence of cis and trans haplotypes; cis and trans haplotype gene dosage; thresholding binding affinities of cis and trans haplotypes to generate categorical features; polynomial and interaction features. The algorithm removed features that were identified as being less relevant than random features in an iterative supervised fashion. This feature selection strategy avoids overfitting when assessing the statistical significance of many features. Features that were identified as being associated with ALVAL were used to train gradient boosted survival analysis machine learning models with a Cox proportional hazards loss function and a regression tree base learner. A gradient boosted survival analysis model iteratively fits regression trees to minimise the negative Cox log-partial likelihood. [46, 47] The Cox log-partial likelihood is at a maximum when the failure and censoring times are fully concordant. Therefore, a model that is trained to minimise the negative of this function (by iteratively fitting regression trees), will attempt to predict concordant results accounting for both failure times and censoring. Regularisation was employed to reduce overfitting on the training data. Nested 5-fold cross-validation (CV) was used on the training data to enable better estimation of generalisation error and reduce model selection bias. Nested CV is preferred over flat CV as it separates the tuning of the model from the estimation of the generalisation error, reducing overfitting on the model selection criterion. [48] Nested CV has shown to be unnecessary for most practical applications except for when the number of hyper-parameters is large [48, 49], as is the case for gradient boosted survival analysis models, hence its use in our validation strategy. Hyper-parameters of both models were optimised using a successive halving random search, which optimises hyper-parameters faster than the traditionally used grid search method. [50, 51] Integrated Brier Score (IBS) was chosen as the scoring function as Applicant's model's primary use was predicting hazard ratios and survival curves and therefore discriminatory ability and calibration were equally important. Cross-validated probability calibration did not yield improvements in IBS and Integrated Calibration Index (ICI). After selecting the best performing model based on the IBS assessed on the training data, the model was then used to predict on the test set. IBS, Uno's c-index, time-dependent AUROC (ROC (t)), and ICI performance statistics were then computed on the test set. Brier score is the mean squared error of the predicted survival probability at a given time point. IBS summarises the Brier score over a particular time interval, representing overall model performance. A low Brier score verifies a high degree of discrimination and calibration. Harrell's c-index is a rank-correlation measure which is an estimate of the probability that for a randomly selected pair of samples, the subject with the lower risk score outlives the subject with the higher risk score. Applicant opted to use Uno's variation of c-index for measuring discriminatory performance, which addresses the overly-optimistic results observed for Harrell's c-index with increasing censoring frequency. [54] Whilst AUROC is equivalent to c-index for binary outcomes, ROC (t) provides a measure of performance for a given time of interest. Applicant used an ROC (t) measure which accounts for censored subjects using the Kaplan-Meier estimator to assess discriminatory performance at discrete time periods.

When a model is tasked with predicting probabilities such as those seen in survival curves, verification of good calibration is important such that predicted probabilities closely follow observed frequencies of survival-ICI may be used to quantify this. ICI is the mean absolute difference between observed and predicted probabilities, weighted by the probability density function of the distribution of predicted probabilities. [56] Applicant used Austin et al.'s adaptation of ICI for survival analysis problems. [57] As the training and testing of the model was disjointed, the performance statistics evaluated on the test set provided measures of how well each model would generalise to unseen data. Confidence intervals were estimated using the Bootstrap method. After completion of performance evaluation, each model was refit on the training and test data and hyper-parameters were retuned. The models were then serialised and integrated into a cloud-hosted pipeline for inference via a web app.

### Results

### Part 1: Investigation of Genetic Associations Through Comparison of Extreme Phenotype Groups (Unit 1)

There was a response rate of around 60% in each of the four subject groups, resulting in a total of 161 subjects who gave saliva or buccal samples for DNA analysis. There were no significant differences in age or sex between responders and non-responders. Subject details and class II haplotype distributions can be seen in Supplementary table 1 and Supplemental Date File 1. There was a bias towards female sex, increased age and THRs in subjects developing ALVAL in response to lower wear. The strongest signals were found with two haplotypes, which had opposing associations with ALVAL. The dominant, significant positive association was seen with DQA1*02:01, DQB1*02:02 and DRB1*07:01. These alleles were increased across all phenotypic subtypes in subjects with prosthetic failure. The alleles were in strong linkage disequilibrium and occurred on one associated haplotype. A protective effect was seen with the alleles DQA1*01:01, DQB1*05:01 and DRB1*01:01.

These alleles, also in strong linkage disequilibrium and occurring on one associated haplotype, were found in significantly higher frequencies in subjects without ALVAL. Class I HLA allele distributions did not differ between the groups.

### Part 2: In Silico Analysis of Peptide-HLA Class II Binding Affinity and Cox Proportional Hazards Modelling (Unit 1)

DQA1*02:01-DQB1*02:02, the haplotype with the strongest positive association to ALVAL, exhibited the strongest binding affinity to the NTS of albumin. DQA1*01:01-DQB1*05:01, the commonly occurring haplotype with the strongest negative association with ALVAL, exhibited one of the weakest binding affinities in the dataset (rank 15 out of 17 haplotypes). Cox proportional hazards modelling, incorporating NTS binding affinities as a continuous measure of DQ haplotype, demonstrated that pre-revision blood Co and Cr concentrations, female sex and greater NTS binding affinity were significantly associated with increased risk of early ALVAL associated failure. These models were consistent using different thresholds of ALVAL (mild and above versus moderate and above (Supplementary Table 2). No relationship was identified between prosthetic failure and the binding affinity values derived from DRB1 molecules. Therefore, a decision was made to expand subject recruitment but focusing solely on DQ molecules.

Part 3: Expansion of Data Set, Recruitment of Subjects from Other Centres and Development and Testing of a Machine Learning Algorithm (Units 1, 2 and 3).

A total of 606 DNA samples, from 397 males and 209 female subjects, were successfully typed. This included 320 subjects from the United Kingdom, 259 from the United States and 27 from Australia. Subject demographics and clinical parameters can be seen in Tables 1 and 2 and Supplementary Table 3.

The clinical details of the training and validation sets are shown in Supplementary Table 4. Supplementary Table 5 shows the results of performance evaluation of the presented models on the test set. Taper-dominated wearing THRs were excluded from the test set for the ALVAL pre-operative model to better fit the clinical context which this model would be exposed to (no MoM THRs are currently implanted in significant numbers-only resurfacings). For all models, the c-index and ROC (t) scores suggested a high degree of discrimination, whilst the IBS indicated good calibration and further backed up the indication of high discriminatory ability. The ICI scores supported the indication of good calibration and showed that at ten years, the weighted mean survival probability error was 1.8% and 3.1% for pre-operative and post-operative ALVAL models respectively (Supplementary Table 5). Supplementary FIGS. 1 and 2 show ALVAL ROC (t) for pre-operative and post-operative models from two to ten years after implantation. The ALVAL pre-operative model peaked in performance at two years after which a similar performance was observed from three to ten years. Similarly consistent performances were observed for the ALVAL post-operative model.

### Survival Analysis Using Total Data Set

Kaplan Meier and Cox proportional survival analyses involving all subjects confirmed the initial single centre results, showing that greater Co (and Cr as the rank correlation between the two elements=0.816, p<0.001) concentrations, female sex, THR prostheses and genotypes with greater NTS binding affinities were significantly associated with greater risk of ALVAL related prosthetic failure (Table 2 and FIGS. 5, 6 and 7).

Applicant has identified that the development of DTH/ALVAL following joint replacement is determined by an interaction between subject sex, genotype and the volume of metal debris generated from a prosthesis. In present day clinical practise, genetic predisposition to DTH is not considered nor tested for in the selection of an orthopaedic implant. Some centres carry out investigations such as skin patch testing or perform lymphocyte proliferation assays to identify subjects who report a “metal allergy” preoperatively. However, these tests have faced continued scrutiny as to their accuracy and true clinical relevance. [62, 63]

Development of ALVAL is Associated with HLA Genotype

Applicant has demonstrated that subjects developing ALVAL to low wearing prostheses possess different frequencies of specific HLA-DQ haplotypes when compared to those who remain asymptomatic at long term follow up. HLA-DQ haplotypes encoding for peptide binding grooves with greater affinity for the NTS of albumin present a higher risk of ALVAL. Albumin is the primary carrier for Co in both synovial fluid and serum. It has several metal ion binding sites, including the N terminal site, which is formed from the first three amino acids of the NTS. Under normal circumstances, the N terminal site is generally occupied by nickel or copper. [64] However, this site can also bind Co ions, particularly when there are changes in the relative concentrations of metal in the surrounding fluid. In addition, the NTS also contains another site with even greater affinity for Co: site B, partially composed of His9 and Asp13. [65]

Metal ions can form complexes with self-proteins held at the peptide binding groove of APCs; these metal peptide complexes can provoke a response from T4 helper cells. However, the presentation of a peptide: MHC complex by an APC does not automatically lead to sensitisation. This is demonstrated by the low ALVAL rates in subjects who possess “higher risk” haplotypes but have low blood metal ion concentrations. Sensitisation requires lymphocyte activation, and for a lymphocyte to become activated, the APC itself must be in an activated form. APCs possess innate pattern recognition receptors (PRRs) (FIG. 2), which, when stimulated, promote activation and migration of APCs from the site of exposure to the draining lymph nodes. This process leads to expansion and survival of metal-reactive memory T cells that circulate throughout the body. Metals can activate PRRs either directly, or indirectly, through the release of reactive oxygen species, the inflammasome pathway [66, 67] or via the induction of necrosis and release of alarmins. An elevation in local metal ion concentrations, therefore, can not only raise the probability of metal: peptide neoantigen presentation, it can also increase the probability of APC activation and thus T cell sensitisation.

Furthermore, an increase in the rate of implant wear can lead to the generation of larger particles which may frustrate effective macrophage phagocytosis, resulting in cell damage, the release of lysosomal products and a local reduction in pH levels.

Albumin peptides, as with other endogenous peptides, are constantly recycled in the body. [70] This recycling commences following pinocytosis or receptor-mediated cellular uptake, when proteins enter the endolysosomal pathway, and are exposed to an increasingly acidic environment (FIG. 3). As the pH drops, peptidases section the ingested proteins into their smaller constituent peptides. Albumin is protected from this endosomal degradation by the binding of neonatal Fc receptor (FcRn), binding which is initiated at pH values below 6.5. [71] However, N terminal albumin sequences 1-24 and 1-26 are some of the first to fragment under mildly acidic conditions, a phenomenon which enables them to act as biomarkers in conditions such as graft versus host disease. Therefore, the NTS could detach from albumin via two mechanisms in different locations: in the synovial fluid itself, or in the endolysosomal pathway, where it is a front runner in the competition to bind with MHC II molecules. Once presented at the cellular membrane, NTS peptides would be exposed to metal ions released from the prosthesis, leading to the formation of metal: peptide complexes (FIG. 3). As is the case with other (largely HLA mediated) autoimmune/autoimmune like diseases, females develop ALVAL more readily than male subjects. This has incorrectly been ascribed this to the tendency for prostheses implanted into females to wear at higher rates. While it is indeed true that MoM hips implanted into females do tend to generate more wear debris, females appear to be more susceptible to ALVAL following exposure to equivalent amounts of metal debris. Accordingly, only females with low wearing prostheses, or genotypes associated with the lowest NTS binding affinity values, were associated with low rates of ALVAL.

MoM THRs have a Higher Risk of ALVAL than Hip Resurfacings.

Applicant has identified that metal debris release from a THR prosthesis is associated with a greater risk of DTH. Applicant speculates that this may be due to differences in the mechanism of metal release—with corrosion playing a more dominant role—and the production of metal species (such as hexavalent chromium [20]) with a greater capacity to activate PRRs. The literature now conclusively shows that MoM THRs fail at higher rates than hip resurfacings, and as a result, they are essentially no longer used in common practise. [6] However, the early failure to appreciate the important distinction between the performance of MoM THRs and hip resurfacings has, some would argue, led to unjustified concerns over the dangers of hip resurfacing, a procedure which has shown good results in young, active males. [6] The present invention makes possible that genotyping can be used to further improve the results of resurfacing, a procedure which has the advantage of allowing the retention of the subject's native proximal femur. Despite this, many surgeons would argue that THRs using modern ceramics and highly cross linked polyethylenes are preferable, given their proven long term survival rates and very low reported rates of DTH. [6]

The results have implications for other types of joint replacements. Almost all commonly used total knee replacements (TKRs) include at least one CoCr component, and revision knee prostheses often incorporate a mixed metal modular junction. Yet, while it is now established that CoCr debris released from MoM hips is of great concern-necessitating specific guidance from orthopaedic societies[17]—there is a lack of consensus as to the clinical significance of metal sensitivity in the field of knee surgery.[75] This may be due to a lack of standardisation in terminology, with the ill-defined condition “allergy” frequently referred to in the literature concerning knee prostheses. [76]

### The Immunogenetics of ALVAL

The Applicant focused on the divergence in the cellular response with reference to the development of ALVAL. We focused specifically on ALVAL for two major clinical reasons, the first being that the relationship between ALVAL and blood metal ion testing appears complex and ion concentrations do not appear to be of reliable diagnostic indicators in ruling the condition in or out. The second is that ALVAL appears to be an important, if not the most important, factor in the development of progressive and irreversible soft tissue damage.

The recruitment of lymphocytes to the area of macrophage driven inflammation lies in the handling and presentation of particulate debris to CD4+ T cells by MHC II molecules on the membranes of APCs. Peptide-MHC binding is a prerequisite for T-cell immunogenicity and multiple studies have shown that there is a strong correlation between MHC peptide binding strength and peptide immunogenicity. Peptides that make stable peptide-MHC complexes accumulate on the cell surface and it has been shown that the total number of peptide-MHC complexes is important for T-cell activation.

When particulate matter is ingested by macrophages, peptide fragments which are produced in the lysosomes compete for the binding grooves of MHC molecules.

Applicant believes that that the antigen presented by APCs to initiate lymphocyte recruitment (the epitope) is a peptide derived from the breakdown of an albumin-Co metalloprotein. Further MHC II genes which differed in frequency between the ALVAL and non-ALVAL subject groups would show greater relative binding affinities for albumin derived peptides. Therefore, given the protection that younger age and male sex appears to confer, testosterone derived peptides might create competition with albumin for these binding sites.

Each subject has four potential DQA1/DQB1 combinations. It is therefore not ideal, when one is investigating the effect of a three-dimensional structure, to study only some of the building blocks in isolation i.e. by simply comparing alleleic frequencies of single genes between subject groups. By calculating theoretical binding affinities, produced a quantitative measure of a subject's genetic makeup which went beyond the classical approach of labelling a subject as being homozygous/heterozygous/lacking in an individual allele.

Applicant discovered that DQ molecules which are particularly suited to bind albumin fragments were significantly associated with the development of ALVAL. Conversely, DQ molecules which bind testosterone fragments with greater affinity were negatively associated with the development of ALVAL. Of particular significance, we believe, was that DQ isoforms in trans had relatively little influence on the statistical modelling compared to the cis combinations. Cis DQ isoforms are more easily formed, more numerous on cell membranes and are thought to be more important for T cell activation.

### Albumin Binding and Antigen Presentation

Following antigen recognition and lymphocyte activation, chemokine release leads to the development of fluid exudates, with greater concentrations of albumin forming in the joint fluid. It is possible therefore that a vicious cycle is set in motion, with lymphocytes sensitised to an antigen which becomes present in ever greater quantities. An increase in the fraction of protein bound metal would also result in greater amounts of metal exiting the joint via the lymphatic system rather than via directly through the synovial membrane, as smaller solutes and ions are capable of doing. Modulating effects of testosterone Sex hormones play an important role in immune modulation. Furthermore, testosterone is present in the synovial fluid of healthy and arthritic joints in concentrations comparable to those of metal ions produced from low wearing MoM devices. A significant fraction of testosterone is albumin bound, meaning that particles digested by macrophages and dendritic cells are likely to contain varying amounts of Co, Cr, albumin and testosterone derived peptides. Levels of gonadal steroids in synovial fluid show an inverse relationship to age. Clearly, the modulation of the immune response by sex hormones is extremely complex. Applicant suggests however that one of the reasons females and older subjects may be more vulnerable to ALVAL might be due to their reduced synovial testosterone concentration and thus reduced competition for MHC binding spaces.

### REFERENCES

1. Pabinger, C. and A. Geissler, Utilization rates of hip arthroplasty in OECD countries. Osteoarthritis and Cartilage, 2014. 22 (6): p. 734-741.
2. Willert, H. G., H. Bertram, and G. H. Buchhorn, Osteolysis in alloarthroplasty of the hip. The role of ultra-high molecular weight polyethylene wear particles. Clin Orthop Relat Res, 1990 (258): p. 95-107.
3. Harris, W. H., The problem is osteolysis. Clin Orthop Relat Res, 1995 (311): p. 46-53.
4. Treacy, R. B., C. W. McBryde, and P. B. Pynsent, Birmingham hip resurfacing arthroplasty. A minimum follow-up of five years. J Bone Joint Surg Br, 2005. 87 (2): p. 167-70.
5. Heisel, C., et al., Ten different hip resurfacing systems: biomechanical analysis of design and material properties. Int Orthop, 2009. 33 (4): p. 939-43.
6. van Lingen, C. P., et al., Sequelae of large-head metal-on-metal hip arthroplasties: Current status and future prospects. I open reviews, 2017. 1 (10): p. 345-353.
7. Davies, A. P., et al., An unusual lymphocytic perivascular infiltration in tissues around contemporary metal-on-metal joint replacements. J Bone Joint Surg Am, 2005. 87.
8. Pandit, H., et al., Pseudotumours associated with metal-on-metal hip resurfacings. J Bone Joint Surg Br, 2008. 90 (7): p. 847-51.
9. Natu, S., et al., Adverse reactions to metal debris: histopathological features of periprosthetic soft tissue reactions seen in association with failed metal on metal hip arthroplasties. Journal of clinical pathology, 2012. 65 (5): p. 409-418.
10. Willert, H. G., et al., Metal-on-metal bearings and hypersensitivity in patients with artificial hip joints. A clinical and histomorphological study. J Bone Joint Surg Am, 2005. 87.
11. Nawabi, D. H., et al., MRI predicts ALVAL and tissue damage in metal-on-metal hip arthroplasty. Clinical orthopaedics and related research, 2014. 472 (2): p. 471-481.
12. G., G., et al., Hip resurfacings revised for inflammatory pseudotumour have a poor outcome. The Journal of Bone and Joint Surgery. British volume, 2009. 91-B (8): p. 1019-1024.
13. Jameson, S. S., et al., The influence of age and sex on early clinical results after hip resurfacing: an independent center analysis. J Arthroplasty, 2008. 23 (6 Suppl 1): p. 50-5.
14. Langton, D. J., et al., Accelerating failure rate of the ASR total hip replacement. J Bone Joint Surg Br, 2011. 93 (8): p. 1011-6.
15. De Smet, K., et al., Metal ion measurement as a diagnostic tool to identify problems with metal-on-metal hip resurfacing. J Bone Joint Surg Am, 2008. 90 Suppl 4: p. 202-8.
16. Hart, A. J., et al., The painful metal-on-metal hip resurfacing. J Bone Joint Surg Br, 2009. 91 (6): p. 738-44.
17. http://www.mhra.gov.uk/home/groups/dts bs/documents/medicaldevicealert/con155767.pdf.
18. Liow, M. H., et al., Metal lon Levels Are Not Correlated With Histopathology of Adverse Local Tissue Reactions in Taper Corrosion of Total Hip Arthroplasty. J Arthroplasty, 2016. 31 (8): p. 1797-802.
19. Langton, D. J., et al., The clinical implications of elevated blood metal ion concentrations in asymptomatic patients with MOM hip resurfacings: a cohort study. BMJ open, 2013. 3 (3): p. e001541.
20. Langton, D., et al., Is the synovial fluid cobalt-to-chromium ratio related to the serum partitioning of metal debris following metal-on-metal hip arthroplasty? Bone & joint research, 2019. 8 (3): p. 146-155.
21. Richeldi, L., R. Sorrentino, and C. Saltini, HLA-DPB1 glutamate 69: a genetic marker of beryllium disease. Science, 1993. 262 (5131): p. 242-4.
22. Lison, D., et al., Experimental research into the pathogenesis of cobalt/hard metal lung disease. Eur Respir J, 1996. 9 (5): p. 1024-8.
23. Budinger, L. and M. Hertl, Immunologic mechanisms in hypersensitivity reactions to metal ions: an overview. Allergy, 2000. 55 (2): p. 108-15.
24. Sinigaglia, F., The molecular basis of metal recognition by T cells. J Invest Dermatol, 1994. 102 (4): p. 398-401.
25. Rosenman, K. D., et al., HLA class II DPB1 and DRB1 polymorphisms associated with genetic susceptibility to beryllium toxicity. Occup Environ Med, 2011. 68 (7): p. 487-93.
26. Griem, P., et al., T cell cross-reactivity to heavy metals: identical cryptic peptides may be presented from protein exposed to different metals. Eur J Immunol, 1998. 28 (6): p. 1941-7.
27. Predki, P. F., et al., Further characterization of the N-terminal copper (II)-and nickel (II)-binding motif of proteins. Studies of metal binding to chicken serum albumin and the native sequence peptide. The Biochemical journal, 1992. 287 (Pt 1) (Pt 1): p. 211-215.
28. Langton, D., et al., Adverse reaction to metal debris following hip resurfacing: the influence of component type, orientation and volumetric wear. The Journal of bone and joint surgery. British volume, 2011. 93 (2): p. 164-171.
29. Sidaginamale, R., et al., Blood metal ion testing is an effective screening tool to identify poorly performing metal-on-metal bearing surfaces. Bone & joint research, 2013. 2 (5): p. 84-95.
30. Langton, D. J., et al., The effect of component size and orientation on the concentrations of metal ions after resurfacing arthroplasty of the hip. J Bone Joint Surg Br, 2008. 90 (9): p. 1143-51.
31. Langton, D., et al., Investigation of taper failure in a contemporary metal-on-metal hip arthroplasty system through examination of unused and explanted prostheses. JBJS, 2017. 99 (5): p. 427-436.
32. Langton, D. J., et al., A comparison study of stem taper material loss at similar and mixed metal head-neck taper junctions. Bone Joint J, 2017. 99-b (10): p. 1304-1312.
33. Langton, D. J., et al., Aseptic lymphocyte-dominated vasculitis-associated lesions are related to changes in metal ion handling in the joint capsules of metal-on-metal hip arthroplasties. Bone Joint Res, 2018. 7 (6): p. 388-396.
34. Reito, A., et al., Prevalence of Failure due to Adverse Reaction to Metal Debris in Modern, Medium and Large Diameter Metal-on-Metal Hip Replacem-nts-The Effect of Novel Screening Methods: Systematic Review and Metaregression Analysis. PLoS One, 2016. 11 (3): p. e0147872.
35. Campbell, P., S. H. Park, and E. Ebramzadeh, Semi-quantitative histology confirms that the macrophage is the predominant cell type in metal-on-metal hip tissues. J Orthop Res, 2021.
36. Dudbridge, F., Likelihood-based association analysis for nuclear families and unrelated subjects with missing genotype data. Hum Hered, 2008. 66 (2): p. 87-98.
37. Langton, D. J., et al., The influence of HLA genotype on the severity of COVID-19 infection. Hla, 2021. 98 (1): p. 14-22.
38. Houdek, M. T., et al., Synovial Fluid Metal lon Levels are Superior to Blood Metal lon Levels in Predicting an Adverse Local Tissue Reaction in Failed Total Hip Arthroplasty. The Journal of Arthroplasty, 2021.
39. Wallis, W. J., P. A. Simkin, and W. B. Nelp, Protein traffic in human synovial effusions. Arthritis Rheum, 1987. 30 (1): p. 57-63.
40. Simkin, P. A. and J. E. Bassett, Pathways of microvascular permeability in the synovium of normal and diseased human knees. J Rheumatol, 2011. 38 (12): p. 2635-42.
41. Lakusta, H. and B. Sarkar, Equilibrium studies of zinc (II) and cobalt (II) binding to tripeptide analogues of the amino terminus of human serum albumin. Journal of Inorganic Biochemistry, 1979. 11 (4): p. 303-315.
42. Jensen, K. K., et al., Improved methods for predicting peptide binding affinity to MHC class II molecules. Immunology, 2018. 154 (3): p. 394-406.
43. Reynisson, B., et al., Improved Prediction of MHC II Antigen Presentation through Integration and Motif Deconvolution of Mass Spectrometry MHC Eluted Ligand Data. J Proteome Res, 2020. 19 (6): p. 2304-2315.
44. Goodfellow, I., Y. Bengio, and A. Courville, Deep learning. 2016: MIT press.
45. Kursa, M. B. and W. R. Rudnicki, Feature Selection with the Boruta Package. 2010, 2010. 36 (11): p. 13.
46. Ridgeway, G. The State of Boosting 1999.
47. COX, D. R., Partial likelihood. Biometrika, 1975. 62 (2): p. 269-276.
48. Cawley, G. C. and N. L. C. Talbot, On Over-fitting in Model Selection and Subsequent Selection Bias in Performance Evaluation. J. Mach. Learn. Res., 2010. 11: p. 2079-2107.
49. Wainer, J. and G. Cawley, Nested cross-validation when selecting classifiers is overzealous for most practical applications. Expert Systems with Applications, 2021. 182: p. 115222.
50. Jamieson, K. and A. Talwalkar. Non-stochastic best arm identification and hyperparameter optimization, in Artificial Intelligence and Statistics. 2016. PMLR.
51. Li, L., et al., Hyperband: A novel bandit-based approach to hyperparameter optimization. The Journal of Machine Learning Research, 2017. 18 (1): p. 6765-6816.
52. Harrell, F. E., Missing data, in Regres41odellingeling strategies. 2001, Springer. p. 41-52.
53. Harrell, F. E., et al., Evaluating the yield of medical tests. Jama, 1982. 247 (18): p. 2543-2546.
54. Uno, H., et al., On the C-statistics for evaluating overall adequacy of risk prediction procedures with censored survival data. Statistics in medicine, 2011. 30 (10): p. 1105-1117.
55. Heagerty, P. J. and P. Saha, SurvivalROC: time-dependent ROC curve estimation from censored survival data. Biometrics, 2000. 56 (2): p. 337-344.
56. Austin, P. C. and E. W. Steyerberg, The Integrated Calibration Index (ICI) and related metrics for quantifying the calibration of logistic regression models. Statistics in medicine, 2019. 38 (21): p. 4051-4065.
57. Peter, C. A., E. H. Frank Jr, and v.K. David, Graphical Calibration Curves and the Integrated Calibration Index (ICI) for Survival Models. Statistics in medicine.
58. Bozic, K. J., et al., The epidemiology of bearing surface usage in total hip arthroplasty in the United States. J Bone Joint Surg Am, 2009. 91 (7): p. 1614-20.
59. 12th Annual Report. National Joint Registry of England and Wales, 2015.
60. Kilb, B. K. J., et al., Frank Stinchfield Award: Identification of the At-risk Genotype for Development of Pseudotumors Around Metal-on-metal THAs. Clin Orthop Relat Res, 2018. 476 (2): p. 230-241.
61. Blowers, P., Immune system involvement in metal hip implant failure. 2015, University of East Anglia.
62. Yang, S., et al., Lymphocyte Transformation Testing (LTT) in Cases of Pain Following Total Knee Arthroplasty: Little Relationship to Histopathologic Findings and Revision Outcomes. JBJS, 2019. 101 (3): p. 257-264.
63. Haddad, S. F., et al., Exploring the Incidence, Implications, and Relevance of Metal Allergy to Orthopaedic Surgeons. J Am Acad Orthop Surg Glob Res Rev, 2019. 3 (4): p. e023.
64. Bal, W., et al., Binding of transition metal ions to albumin: Sites, affinities and rates. Biochimica et Biophysica Acta (-BA)-General Subjects, 2013. 1830 (12): p. 5444-5455.
65. Mothes, E. and P. Faller, Evidence that the principal Coll-binding site in human serum albumin is not at the N-terminus: implication on the albumin cobalt binding test for detecting myocardial ischemia. Biochemistry, 2007. 46 (8): p. 2267-74.
66. Caicedo, M. S., et al., Increasing both CoCrMo-alloy particle size and surface irregularity induces increased macrophage inflammasome activation in vitro potentially through lysosomal destabilization mechanisms. J Orthop Res, 2013. 31 (10): p. 1633-42.
67. Yazdi, A. S., K. Ghoreschi, and M. Röcken, Inflammasome Activation in Delayed-Type Hypersensitivity Reactions. Journal of Investigative Dermatology, 2007. 127 (8): p. 1853-1855.
68. McKee, A. S., et al., MyD88 dependence of beryllium-induced dendritic cell trafficking and CD4+T-cell priming. Mucosal Immunol, 2015. 8 (6): p. 1237-47.
69. Perino, G., et al., The contribution of the histopathological examination to the diagnosis of adverse local tissue reactions in arthroplaIEFORT Open Reviews, 2021. 6 (6): p. 399-419.
70. Pete-s, T., 6-Clinical Aspects: Albumin in Medicine, in All About Albumin, T. Peters, Editor. 1995, Academic Press: San Diego. p. 251-284.
71. Chaudhury, C., et al., The major histocompatibility complex-related Fc receptor for lgG (FcRn) binds albumin and prolongs its lifespan. Journal of Experimental Medicine, 2003. 197 (3): p. 315-322.
72. Yang, J., et al., Mass spectrometric characterization of limited proteolysis activity in human plasma samples under mild acidic conditions. Methods, 2015. 89: p. 30-7.
73. Langton, D., et al., Accelerating failure rate of the ASR total hip replacement. The Journal of bone and joint surgery. British volume, 2011. 93 (8): p. 1011-1016.
74. Gavin, I. M., et al., Identification of human cell responses to hexavalent chromium. Environ Mol Mutagen, 2007. 48 (8): p. 650-7.
75. Innocenti, M., et al., Metal hypersensitivity after knee arthroplasty: fact or fiction? Acta bio-medica: Atenei Parmensis, 2017. 88 (2S): p. 78-83.
76. Saccomanno, M. F., et al., Allergy in total knee replacement surgery: Is it a real problem? World J Orthop, 2019. 10 (2): p. 63-70.
77. Kretzer, J. P., et al., Wear in total knee ar-roplasty-just a question of polyethylene?: Metal ion release in total knee arthroplasty. Int Orthop, 2014. 38 (2): p. 335-40.
78. Arnholt, C. M., et al., Corrosion Damage and Wear Mechanisms in Long-Term Retrieved CoCr Femoral Components for Total Knee Arthroplasty. J Arthroplasty, 2016. 31 (12): p. 2900-2906.
79. Reiner, T., et al., Blood Metal lon Release After Primary Total Knee Arthroplasty: A Prospective Study. Orthop Surg, 2020. 12 (2): p. 396-403.
80. Luetzner, J., et al., Serum metal ion exposure after total knee arthroplasty. Clin Orthop Relat Res, 2007. 461: p. 136-42.
81. Savarino, L., et al., The potential role of metal ion release as a marker of loosening in patients with total knee replacement: a cohort study. J Bone Joint Surg Br, 2010. 92 (5): p. 634-8.
82. Kurmis, A. P., et al., Pseudotumors and High-Grade Aseptic Lymphocyte-Dominated Vasculitis-Associated Lesions Around Total Knee Replacements Identified at Aseptic Revision Surgery: Findings of a Large-Scale Histologic Review. J Arthroplasty, 2019. 34 (10): p. 2434-2438.
83. Crawford, D. A., et al., Impact of perivascular lymphocytic infiltration in aseptic total knee revision. Bone Joint J, 2021. 103-b (6 Supple A): p. 145-149.
84. Wylde, V., et al., Chronic pain after total knee arthlsty. EFORT open reviews, 2018. 3 (8): p. 461-470.
85. Sidaginamale, R. P., et al., Blood metal ion testing is an effectivescreening tool to identify poorly performing metal-on-metal bearing surfaces. Bone Joint Res, 2013. 2 (5): p. 84-95.
86. Wysocki, T., M. Olesioska, and A. Paradowska-Gorycka, Current Understanding of an Emerging Role of HLA-DRB1 Gene in Rheumatoid Arthritis-From Research to Clinical Practice. Cells, 2020. 9 (5).
87. Sollid, L. M., The roles of MHC class II genes and post-translational modification in celiac disease. Immunogenetics, 2017. 69 (8-9): p. 605-616.

The invention claimed is:

1. A method of screening a subject to determine the likelihood of adverse response to metal debris (ARMD) or adverse local tissue reaction (ALTR), said method comprising the steps of:
determining whether the subject has one or more of the following markers associated with an adverse response wherein the marker comprises one or more from the following table:

| SNP | Gene | Protein |
|---|---|---|
| GSA-rs35269015 | MCC | relating to the function and/or |
| | | expression of MCC |
| GSA-rs68164453 | Intragenic | relating to the function and/or |
| | | expression of LOC105374976 |
| GSA-rs10985824 | u/s RABGAP1 | relating to the function and/or |
| | | expression of RABGAP1 |
| GSA-rs190079656 | ADAMTSL3 | relating to the function and/or |
| | | expression of the ADAMTSL3 |
| GSA-rs117972780 | OR1L4 | relating to the function and/or |
| | | expression of OR1L4 |
| GSA-rs45539538 | CYP24A1 | relating to the function and/or |
| | | expression of CYP24A1 |
| GSA-rs79208627 | LOC101927964 | relating to the function and/or |
| | | expression of LOC101927964 |
| GSA-rs976508 | PIGN | relating to the function and/or |
| | | expression of PIGN |
| GSA-rs76147841 | GABBR2 | relating to the function and/or |
| | | expression of GABBR2 |
| GSA-rs1930426 | u/s RABGAP1 | relating to the function and/or |
| | | expression of GABBR2 |
| rs56177975 | d/s TGFB2 | relating to the function and/or |
| | | expression of TGF2 |
| rs1392779 | between INPP4B | (relating to the function and/or |
| | and IL5 | expression of INPP4B and IL5 |
| rs13145571 | SULT1E1 | relating to the function and/or |
| | | expression of SULT1E1 |
| rs10790129 | intergenic | |
| rs2304968 | SLFN12L | relating to the function and/or |
| | | expression of SLFN12L |
| rs4798850 | RAB31 | relating to the function and/or |
| | | expression of RAB31 |
| rs57039293 | UMAD1 | Relating to the function and/or |
| | | expression of UMAD1 |
| rs79000696 | CYP24A1 | Relating to the function and/or |
| | | expression of CYP24A1 |

and optionally,
HLA-DRB1*01, DRB1*04, DRB1*10 (18, 19) DQA1*05: 01/DQB1*02:01 HLA-DRB1*07, HLA-DRB1*0103, HLA-DRB1*04 and HLA-DRB3*0301. (20),
wherein the presence of one or more of the markers associated with an adverse response is indicative of an increased likelihood of an adverse response in a subject.

2. The method according to claim 1, further comprising contacting in vitro a subject's biological sample with means to detect the presence and/or absence of a marker associated with an adverse response wherein the marker comprises one or more of the following SNPs: rs17090828, GSA-rs1930426, GSA-rs79208627, GSA-rs45539538, GSA-rs76147841, GSA-rs117972780, rs4798850, rs11974031, rs9301947, GSA.rs17716559 PIGN, rs12248205, rs1392779, rs13145571, GSA-rs35269015, GSA-rs68164453, GSA-rs10985824, GSA-rs190079656, GSA-rs76147841, GSA-rs117972780, GSA-rs79208627, GSA-rs976508, GSA-rs35269015, GSA-rs76147841, GSA-rs1930426, and GSA-rs45539538; and
determining whether the subject has the marker associated with an adverse response.

3. The method according to claim 1, wherein the marker associated with adverse reaction to metal debris (ARMD) and/or adverse local tissue reaction (ALTR) further comprises, a subject's age, a subject's sex, vitamin D concentrations, and/or blood concentration of cobalt, chromium, titanium, and/or zirconium.

4. The method according to claim 1, wherein the marker associated with adverse reaction to metal debris (ARMD) and/or adverse local tissue reaction (ALTR) further comprises any one or more of the following:
HLA-DRB1*01, DRB1*04, DRB1*10 (18, 19), DQA1*05: 01/DQB1*02:01, HLA-DRB1*07, HLA-DRB1*0103, HLA-DRB1*04, HLA-DRB3*0301. (20), DQA1*02:01, DQB1*02:02, and DRB1*07:01.

5. The method according to claim 1, wherein the biological sample comprises a solid and/or fluid sample.

6. The method according to claim 5, wherein
the fluid sample comprises one or more of the following: a blood sample, saliva, skin cells, or a blood extract sample.

7. A method of screening a compound or a therapeutic agent for use in the treatment of adverse reaction to metal debris (ARMD) and/or adverse local tissue reaction (ALTR) response to an implant or use in preventing or ameliorating the likelihood of ARMD or ALTR in a subject, said method comprising the steps of contacting the compound or therapeutic agent with albumin-cobalt metalloprotein to determine if binding occurs or determining whether the compound or therapeutic agent interferes with an MHC mediated immune response.

8. The method according to claim 1, wherein the adverse reaction is ALVAL.

9. The method according to claim 1, wherein said method is performed prior to, during, and/or after the subject is subjected to an implant procedure.

10. The method according to claim 8, wherein following the implant procedure, blood metal ions are measured.

11. The method according to claim 9, wherein the metal ions comprise any one or more of the following: Co, Cr, Zr, Ti, nickel, and aluminium.

12. The method according to claim 3, wherein the biological sample comprises skin cells from the buccal cavity.

13-14. (canceled)

15. A kit or device for screening a subject to determine the likelihood of adverse response to metal debris (ARMD) or adverse local tissue reaction (ALTR), comprising: reagents or means for determining whether an individual has any of the following SNPs: rs17090828, GSA-rs1930426, GSA-rs79208627, GSA-rs45539538, GSA-rs76147841, GSA-rs117972780, rs4798850, rs11974031, rs9301947, GSA.rs17716559 PIGN, rs12248205, rs1392779, rs13145571, GSA-rs35269015, GSA-rs68164453, GSA-rs10985824, GSA-rs190079656, GSA-rs76147841, GSA-rs117972780, GSA-rs79208627, GSA-rs976508, GSA-rs35269015,
GSA-rs76147841, GSA-rs1930426 and GSA-rs45539538; and optionally reagents or means for determining vitamin D status of a subject; and directions for determining the likelihood of adverse response to metal debris (ARMD) or adverse local tissue reaction (ALTR) based on the presence of any of the SNPs and optionally, the vitamin D status of the subject.

16. The kit or device according to claim 15, wherein the reagents for determining whether a subject has any of the marker SNPs comprises at least one set of oligonucleotide primers.

17. (canceled)

18. The kit or device according to claim 15, further comprising a means for displaying output data.

19. The kit or device according to claim 18, wherein the output data comprises an indication of one or more of the following: whether an individual has at least one of the markers associated with an adverse response; an individual's vitamin D status; the risk of an individual developing an adverse response.

20. The kit or device according to claim 15, further comprising
reagents for determining whether the individual has any one or more of the following markers: HLA-DRB1*01, DRB1*04, DRB1*10 (18, 19), DQA1*05: 01/DQB1*02:01, HLA-DRB1*07, HLA-DRB1*0103, HLA-DRB1*04, HLA-DRB3*0301. (20), DQA1*02:01, DQB1*02:02 and DRB1*07:01;
reagents for determining vitamin D status of an individual; and
directions for determining the likelihood of adverse response to metal debris (ARMD) or adverse local tissue reaction (ALTR), based on the presence of any one or more of the markers and the vitamin D status of the individual.
